(19) United States
(12) Patent Application Publication
(10) Pub. No.: US 2004/0260343 A1
(43) Pub. Date: Dec. 23, 2004

(54) T-type bone anchor

(76) Inventor: Walter J. Leclair, Shrewsbury, MA (US)

Correspondence Address: LAHIVE & COCKFIELD, LLP., 28 STATE STREET, BOSTON, MA, 02109, US

(21) Appl. No.: 10/766,501

(22) Filed: Jan. 27, 2004

Related U.S. Application Data
(60) Provisional application No. 60/444,865, filed on Feb. 4, 2003.

(51) Int. Cl. A61B 17/04

(52) U.S. Cl. 606/232; 606/72

(57) ABSTRACT

A T-type bone anchor for attaching a structure, such as a braided cable, to bone includes an end unit attached in a T configuration to surgical cable. The anchor and cable can be made of a biocompatible material such as stainless steel, titanium, titanium alloy, or other metals. The end unit enables distribution of pull forces over a large cross section of bone. The T-type arrangement of the end unit also enables insertion of the T-type bone anchor through a small hole relative to cable diameter. The cable portion of the T-type bone anchor enables folding of the T-type bone anchor to facilitate insertion, flexibility, and resistance to fatigue failure. The cable can be coupled to orthopedic devices in a conventional manner. The T-type bone anchor and attached cable can be folded onto the end unit portion allowing packing into a conduit or needle for delivery into the bone.

### RELATED APPLICATION

This application claims priority to and the benefit of U.S. Provisional application No. 60/444,865, filed on Feb. 4, 2003, which is expressly and entirely incorporated herein by reference.

### FIELD OF THE INVENTION

The present invention relates to an orthopedic bone anchor, and more particularly to a T-type bone anchor, and corresponding method of use.

### BACKGROUND OF THE INVENTION

In the practice of orthopedic surgery, there is often a need to secure surgical cable to bone in order to facilitate repair and reconstruction of the bone. Present methods of fixation utilize anchors that use either screw type fixation, or barbs of various types to hold the anchors in place. Surgical wire or suture is then attached to the anchor, or is pre-attached by the manufacturer.

Conventional anchors are expensive to produce. In addition, conventional anchors distribute the pull out forces over a small area of bone, making the strength of the bone a significant factor in the strength of the fixation. Further, conventional anchors are of large diameter relative to the suture or cable utilized, and are difficult to attach to surgical cable. Surgical cable most often consists of small diameter braided cable formed of stainless steel, titanium, and other metals. The braided cable has significant advantages in orthopedic surgery over solid wire in strength, fatigue resistance, and ease of coupling to other orthopedic devices.

### SUMMARY OF THE INVENTION

There is a need in the art for a bone anchor that is inexpensive to produce, distributes pull out forces over a relatively large cross section of bone, can be inserted through a small diameter hole, and has surgical cable attached thereto. The present invention provides a solution to address this need.

In accordance with one embodiment of the present invention, an orthopedic anchor includes a biocompatible end unit segment. A biocompatible cable couples with the end unit segment generally forming a “T” shape. The end unit segment folds against the cable for both the end unit segment and cable to fit through a hole, and the end unit segment can return to the “T” shape after passing through the hole to anchor the cable.

In accordance with aspects of the present invention, the orthopedic anchor has sufficient strength to withstand foreseeable pull forces experienced during use as an anchor for orthopedic implantation. The end unit segment can have a generally cylindrical shape. The cable can be a braided cable.

In accordance with further aspects of the present invention, the cable couples with the end unit using at least one of a weld, a thermal bond, an adhesive, and a mechanical coupling. The orthopedic anchor can be formed at least partially of at least one of stainless steel and titanium.

In accordance with further aspects of the present invention, the orthopedic anchor can be configured to fit within a delivery conduit when the end unit segment is folded against the cable for implantation through the hole.

In accordance with one embodiment of the present invention, an orthopedic anchor includes a biocompatible end unit segment. A biocompatible cable couples with the end unit segment to generally form a “T” shape. The end unit segment is foldable against the cable to fit within a delivery conduit for delivery of the orthopedic anchor through a hole, and the end unit segment can return to the “T” shape after implantation.

In accordance with one embodiment of the present invention, an orthopedic anchor means includes a biocompatible end unit means. A biocompatible cable means couples with the end unit segment generally forming a “T” shape. The end unit means folds against the cable means for both the end unit means and cable means to fit through a hole, and the end unit means can return to the “T” shape after passing through the hole to anchor the cable means.

In accordance with aspects of the present invention, the orthopedic anchor has sufficient strength to withstand foreseeable pull forces experienced during use as an anchor for orthopedic implantation. The end unit means can have a generally cylindrical shape. The cable means can be a braided cable.

In accordance with aspects of the present invention, the cable means couples with the end unit using at least one of a weld, a thermal bond, an adhesive, and a mechanical coupling. The orthopedic anchor means is formed at least partially of at least one of stainless steel and titanium.

In accordance with aspects of the present invention, the orthopedic anchor means is configured to fit within a delivery conduit when the end unit means is folded against the cable means for implantation through the hole.

### BRIEF DESCRIPTION OF THE DRAWINGS

The present invention will become better understood with reference to the following description and accompanying drawings, wherein:

FIG. 1 is a diagrammatic illustration of a T-type bone anchor, according to one aspect of the present invention;

FIG. 2 is a diagrammatic illustration of the T-type bone anchor within a delivery conduit, according to one aspect of the present invention; and

FIG. 3 is a perspective illustration of the T-type anchor implanted in a bone, according to one aspect of the present invention.

### DETAILED DESCRIPTION

An illustrative embodiment of the present invention relates to a T-type bone anchor for attaching a structure, such as a braided cable, to bone. The T-type bone anchor includes an end unit attached in a T configuration to surgical cable. The anchor and cable can be made of a biocompatible material such as stainless steel, titanium, titanium alloy, or other metals. The end unit enables distribution of pull forces over a large cross section of bone. The T-type arrangement of the end unit also enables insertion of the T-type bone anchor through a small hole relative to cable diameter. The cable portion of the T-type bone anchor enables folding of the T-type bone anchor to facilitate insertion, flexibility, and resistance to fatigue failure. The cable can be coupled to orthopedic devices in a conventional manner. The T-type bone anchor and attached cable can be folded onto the end unit portion allowing packing into a conduit or needle for delivery into the bone.

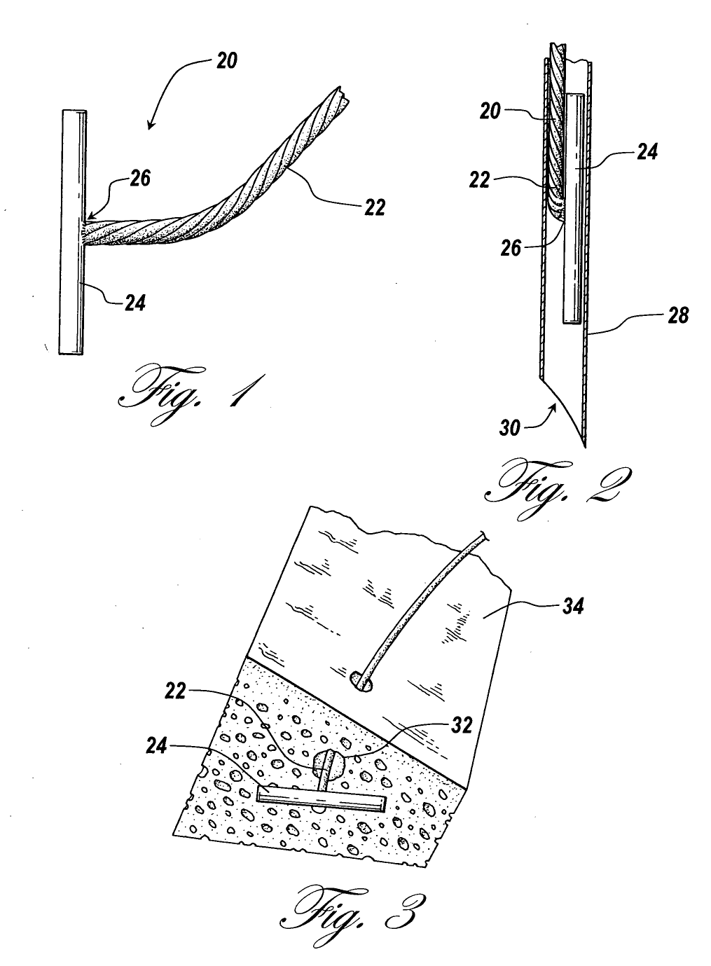

FIGS. 1 through 3, wherein like parts are designated by like reference numerals throughout, illustrate an example embodiment of a T-type bone anchor according to the present invention. Although the present invention will be described with reference to the example embodiment illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

FIG. 1 is a diagrammatic illustration of a T-type bone anchor 20 in accordance with the present invention. The T-type bone anchor 20 includes a cable 22 coupled with a end unit 24. The cable can be braided, or otherwise maintain sufficient strength and flexibility to perform the functions of the present invention. For use as a bone anchor, the cable 22 can be a surgical braided cable.

The cable 22 couples with the end unit 24 at joint 26. The joint 26 can be formed with a weld, thermal bonding, adhesive, or mechanical coupling (e.g, wrapping the cable 22 around the end unit 24), or with another joining method as understood by one of ordinary skill in the art. The resulting joint 26 must be sufficiently strong to both withstand any pull forces exerted by the orthopedic function of the anchor, and withstand being folded prior to delivery as discussed below without fracturing.

The end unit 24 is shown as a generally cylindrical shaft, which can be solid. However, one of ordinary skill in the art will appreciate that the end unit 24 can have a number of different shapes. The general function of the end unit 24 is to couple with the cable 22, as described, have an appropriate shape for sliding through relatively small spaces (such as a hole drilled through a bone or an orthopedic implant) and to have sufficient strength to withstand an anchoring pull force and distribute the pull force across a relatively larger surface area. As mentioned previously, the conventional surgical cable anchor distributes any anchoring pull force exerted on the cable 22 over a relatively small cross-section or surface area of bone. As such, if there are any weaknesses in the particular portion of bone in which the conventional cable anchor is mounted, the anchor can pull out of the bone. However, the shape of the end unit 24 is such that any anchoring pull forces are distributed across a relatively larger surface area, thus resulting in a smaller force per unit measure of area applied to the bone or orthopedic implant. By distributing the force across the larger surface area, the amount of force that can be applied to the T-type bone anchor 20 is greatly increased relative to the convention cable anchor. Accordingly, the shape of the end unit 24 must maintain a sufficient length to effectively distribute any foreseeable anchoring pull force applied to the cable 22.

Furthermore the end unit 22, must likewise be made of a material that is able to withstand any foreseeable pull force exerted on the cable 22. Accordingly, the end unit 22 can be made of biocompatible materials including but not limited to stainless steel, titanium, and/or titanium alloy. The material must maintain sufficient strength to withstand any pull forces applied thereto in a common implantation arrangement without fracturing or bending, and while also being biocompatible (such that the material does not cause detrimental effects if implanted into a patient).

The T-type bone anchor 20 can be implanted using a number of different methods. For example, FIG. 2 is a diagrammatic illustration of the T-type bone anchor 20 disposed within a delivery conduit or needle 28 in accordance with one example delivery or implantation method. The T-type bone anchor 20 bends at the joint 26 in the manner shown to fit within the needle 28. The needle 28 is then positioned through the particular structure to which the anchor is to be fixed. More specifically, the needle can pass through a hole 32 in a bone 34 (see FIG. 3) or other body structure or tissue. Alternatively, the needle 28 can pass through or form a hole in an orthopedic prosthesis. Once the needle 28 is in place, the T-type bone anchor 20 is expelled from the needle 28 through the needle tip 30. Upon exiting the needle 28, the end unit 24 can un-bend (i.e., return to the “T” shape), thus creating an anchor effect.

Likewise, a clinical user of the T-type bone anchor 20 can bend the T-type bone anchor at the joint 26 to fit through a hole in the particular structure to which the T-type bone anchor 20 is to be fixed. More specifically, the clinical user can bend the T-type bone anchor 20 at the joint 26, and pass the end unit 24 through the hole 32 in the bone 34 (see FIG. 3) or other body structure or tissue. Once the end unit 24 passes completely through the hole 32, the end unit 24 can un-bend (i.e., return to the “T” shape), thus creating an anchor effect.

FIG. 3 is a perspective representation of the T-type bone anchor 20 fixed in anchored position in the hole 32 drilled through the bone 34. As previously described, the end unit 24 is returned to its original “T”-shaped position. As such, if a pull force is exerted on the cable 22, the force is distributed across the bone or tissue for the length of the end unit 24.

The T-type bone anchor 20 of the present invention is relatively inexpensive to manufacture. Each of the components of the T-type bone anchor 20 (e.g., the end unit 24 and the cable 22) can be varied in size and dimension to accommodate different anchoring arrangements. For example, the end unit 24 can have different length, width, and/or cross-sectional shape. The cable 22 can have different braid configurations, different lengths and widths, and be rated to handle different pull forces.

Suitable materials for forming the T-type bone anchor 20 include stainless steel, titanium, and/or titanium alloy. The material must maintain sufficient strength to withstand any pull forces applied thereto in a common implantation arrangement, while also being able to bend to fit within the needle 28 without fracturing.

The use of the end unit 24 enables the distribution of any pull force exerted on the cable 22 across a wider area of the bone relative to conventional suturing methods. The ability of the cable 22 to fold without failure enables folding of the T-type bone anchor 20 and insertion through a delivery conduit such as the needle 28 illustrated. Once tension is applied to the cable 22, the end unit 24 self-deploys to create the desired “T” shape and anchor the cable 22.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the disclosed invention is reserved.

What is claimed is:

1. An orthopedic anchor, comprising:
a biocompatible end unit segment; and
a biocompatible cable coupled with the end unit segment generally forming a “T” shape;
wherein the end unit segment folds against the cable for both the end unit segment and cable to fit through a hole, and the end unit segment can return to the “T” shape after passing through the hole to anchor the cable.

2. The orthopedic anchor of claim 1, wherein the orthopedic anchor has sufficient strength to withstand foreseeable pull forces experienced during use as an anchor for orthopedic implantation.

3. The orthopedic anchor of claim 1, wherein the end unit segment comprises a generally cylindrical shape.

4. The orthopedic anchor of claim 1, wherein the cable comprises a braided cable.

5. The orthopedic anchor of claim 1, wherein the cable couples with the end unit using at least one of a weld, a thermal bond, an adhesive, and a mechanical coupling.

6. The orthopedic anchor of claim 1, wherein the orthopedic anchor is formed at least partially of at least one of stainless steel and titanium.

7. The orthopedic anchor of claim 1, wherein the orthopedic anchor is configured to fit within a delivery conduit when the end unit segment is folded against the cable for implantation through the hole.

8. An orthopedic anchor, comprising:
a biocompatible end unit segment; and
a biocompatible cable coupled with the end unit segment to generally form a “T” shape;
wherein the end unit segment is foldable against the cable to fit within a delivery conduit for delivery of the orthopedic anchor through a hole, and the end unit segment can return to the “T” shape after implantation.

9. The orthopedic anchor of claim 8, wherein the orthopedic anchor has sufficient strength to withstand foreseeable pull forces experienced during use as an anchor for orthopedic implantation.

10. The orthopedic anchor of claim 8, wherein the end unit segment comprises a generally cylindrical shape.

11. The orthopedic anchor of claim 8, wherein the cable comprises a braided cable.

12. The orthopedic anchor of claim 8, wherein the cable couples with the end unit using at least one of a weld, a thermal bond, an adhesive, and a mechanical coupling.

13. The orthopedic anchor of claim 8, wherein the orthopedic anchor is formed at least partially of at least one of stainless steel and titanium.

14. An orthopedic anchor means, comprising:
a biocompatible end unit means; and
a biocompatible cable means coupled with the end unit segment generally forming a “T” shape;
wherein the end unit means folds against the cable means for both the end unit means and cable means to fit through a hole, and the end unit means can return to the “T” shape after passing through the hole to anchor the cable means.

15. The orthopedic anchor means of claim 14, wherein the orthopedic anchor has sufficient strength to withstand foreseeable pull forces experienced during use as an anchor for orthopedic implantation.

16. The orthopedic anchor means of claim 14, wherein the end unit means comprises a generally cylindrical shape.

17. The orthopedic anchor means of claim 14, wherein the cable means comprises a braided cable.

18. The orthopedic anchor means of claim 14, wherein the cable means couples with the end unit using at least one of a weld, a thermal bond, an adhesive, and a mechanical coupling.

19. The orthopedic anchor means of claim 14, wherein the orthopedic anchor means is formed at least partially of at least one of stainless steel and titanium.

20. The orthopedic anchor means of claim 14, wherein the orthopedic anchor means is configured to fit within a delivery conduit when the end unit means is folded against the cable means for implantation through the hole.
